(19) United States
(12) Patent Application Publication
(10) Pub. No.: US 2025/0288353 A1
(43) Pub. Date: Sep. 18, 2025

(54) INTEGRATED LASER, WAVELENGTH CONTROL METHOD AND WEARABLE MEDICAL DEVICE

(71) Applicants: SILITH TECHNOLOGY (SUZHOU) CO., LTD., Shanghai (CN); The Second Affiliated Hospital of Zhejiang University School of Medicine, Zhejiang (CN)

(72) Inventors: Zhexuan ZHENG, Shanghai (CN); Nanfei ZHU, Shanghai (CN); Genping GUO, Shanghai (CN); Ke ZHANG, Shanghai (CN)

(73) Assignees: SILITH TECHNOLOGY (SUZHOU) CO., LTD., Shanghai (CN); The Second Affiliated Hospital of Zhejiang University School of Medicine, Zhejiang (CN)

(21) Appl. No.: 19/026,380

(22) Filed: Jan. 17, 2025

(30) Foreign Application Priority Data
Mar. 18, 2024 (CN) 202410302473.0

(51) Int. Cl. A61B 18/20

(52) U.S. Cl. CPC A61B 18/20; A61B 2018/205547

(57) ABSTRACT

Present application relates to field of lasers, provides an integrated laser and a wavelength control method, integrated laser includes a light source, a resonant cavity and an annular mirror; light source is connected with a head end of first optical waveguide through a first coupler; a tail end of first optical waveguide is connected with a second coupler; second coupler is connected with a first end of resonant cavity; second coupler is also provided with a light output end which is used for outputting light waves input by resonant cavity to an object; second end of resonant cavity is connected with a third coupler; third coupler is connected with annular mirror through a second optical waveguide; at least one of resonant cavity, first optical waveguide and second optical waveguide is made of phase-change device. Integrated laser is used for outputting laser of which wavelength can be continuously tuned.

### CROSS-REFERENCE TO RELATED APPLICATION

This application claims the priority benefit of China application serial no. 202410302473.0, filed on Mar. 18, 2024. The entirety of the above-mentioned patent application is hereby incorporated by reference herein and made a part of this specification.

### BACKGROUND

### Technical Field

The present application relates to the technical field of lasers, in particular to an integrated laser and a wavelength control method.

### Description of Related Art

An imaging technique in vivo can provide indispensable physiological and case information for medical clinical diagnosis and design of therapeutic regimens. Currently, an optical imaging technique in vivo, due to a plurality of characters including non-ionizing, non-destructive and non-invasive, is considered a safe and reliable diagnostic method, being able to provide important information for various biomedical applications.

An output wavelength of an existing laser is a fixed value, it requires to switch to a plurality of different optical devices to achieve an output wavelength adjustment, since an output wavelength variation achieved by this solution cannot be continuous, it is difficult to obtain an output wavelength required accurately, causing accuracy during aiming a target tissue be insufficient. It is possible to injure a normal tissue around accidentally, causing a side effect increased, or due to the target tissue cannot be aimed, causing a treatment effect poor. Therefore, a novel integrated laser and a wavelength control method is needed urgently to improve the problem stated above.

### SUMMARY

The purpose of the present application is providing an integrated laser and a wavelength control method, configured to output a laser having a wavelength continuously tunable.

On a first aspect, the present application provides an integrated laser, comprising a light source, a resonant cavity, and an annular mirror; the light source is connected to a head end of a first optical waveguide through a first coupler; a tail end of the first optical waveguide is connected to a second coupler; the second coupler is connected to a first end of a single-ring shaped micro-ring resonant cavity; the second coupler further has a light output end arranged, configured to output a light wave input from the resonant cavity to an object; a second end of the single-ring shaped micro-ring resonant cavity is connected with a third coupler; the third coupler is connected with the annular mirror through a second optical waveguide; the resonant cavity comprises the single-ring shaped micro-ring resonant cavity, the first coupler, the second coupler, the third coupler, the first optical waveguide, and the second optical waveguide; the single-ring shaped micro-ring resonant cavity comprises a first semi-circular cavity and a second semi-circular cavity; both the first semi-circular cavity and the second semi-circular cavity are made of a phase-change device; at least one of the first optical waveguide and the second optical waveguide has a portion configured as a phase-change device; the phase-change device is an optical phase-change material; when the first optical waveguide comprises the phase-change device, a waveguide core of the first optical waveguide is connected directly with the phase-change device, so a transmitted light passes through the optical phase-change material directly; when the second optical waveguide comprises the phase-change device, a waveguide core of the second optical waveguide is connected directly with the phase-change device, so a transmitted light passes through the optical phase-change material directly; the waveguide core is made of a silicon nitride material.

The present application has the following beneficial effects: the resonant cavity in the present application is a single-ring shaped micro-ring resonant cavity, comprising the first semi-circular cavity and the second semi-circular cavity; each of the first semi-circular cavity and the second semi-circular cavity is configured as a phase-change device; at least one of the first optical waveguide and the second optical waveguide has a portion configured as a phase-change device, it is beneficial for changing a refractive index of an output light wave from the laser through optical phase-change, and a wavelength of the output light wave is continuously tunable along with the refractive index, so as to obtain an output light wave with a required wavelength, which is beneficial to improving a precision of aiming a target tissue, avoiding an accidental injury to surrounding normal tissues, and reducing a side effect.

Preferably, the third coupler is further connected to a light absorber, configured to absorb a portion of the light wave output from the resonant cavity to eliminate reflected light, and another portion of the light wave output from the resonant cavity is input into the annular mirror by the second optical waveguide.

Preferably, the third coupler is further connected to a unidirectional optical waveguide, and the unidirectional optical waveguide allows a light wave to propagate in a direction away from the third coupler only.

Preferably, the annular mirror is configured to transmit the light wave input into the annular mirror back to the second optical waveguide; and the resonant cavity is configured to transmit the light wave input from the second optical waveguide to the second coupler.

Preferably, a lattice state of the phase-change device after being heated changes with a cooling speed, so that the refractive index of the phase-change device after being cooled down changes to a preset fixed value.

Preferably, the resonant cavity is configured as a non-linear closed-loop resonant cavity.

Preferably, the annular mirror comprises a passive optical device and a loopback optical waveguide; the passive optical device has at least three terminals arranged, configured to split one light beam into two light beams; and the loopback optical waveguide is connected to two of the at least three terminals of the passive optical device.

On a second aspect, the present application provides a wavelength control method for an integrated laser, configured to control the integrated laser according to any one of the first aspect, comprising: S1, obtaining a refractive index and a physical length of each optical device, and calculating an optical path length of a light wave from a light source to a light output end; the optical device comprises a first optical waveguide, a second optical waveguide, a resonant cavity and an annular mirror; S2, calculating a current output wavelength of the laser according to the optical path length in present; S3, controlling a heating part to heat up the optical device according to a heating instruction; and S4, controlling a cooling part to cool down the optical device according to a cooling instruction, while a cooling speed is adjustable.

Preferably, an output wavelength λ of the light wave satisfies:

$$m * λ = L$$

wherein L is the optical path length of the light wave from the light source to the light output end, and m is a positive integer.

Preferably, the optical path length L satisfies:

$$L = {n}_{1} {l}_{1} + {n}_{2} {l}_{2} + … + {n}_{i} {l}_{i}$$

wherein i is a sequence number of each optical device in the laser; nᵢ is a refractive index of an i-th optical device; and lᵢ is a physical length of the i-th optical device.

On a third aspect, the present application provides a wearable medical device, comprising the integrated laser according to any one of the first aspect.

### BRIEF DESCRIPTION OF THE DRAWINGS

FIG. 1 illustrates a schematic structural diagram on a laser with a prism system in the prior art;

FIG. 2 illustrates a schematic structural diagram on a silicon photonics integrated laser in the prior art;

FIG. 3 illustrates a schematic structural diagram on an integrated laser provided by the present application;

FIG. 4 illustrates a schematic structural diagram on a first optical waveguide provided by the present application;

FIG. 5 illustrates a schematic structural diagram on an annular mirror provided by the present application;

FIG. 6 illustrates a schematic flowchart on a wavelength control method for an integrated laser provided by the present application;

FIG. 7 illustrates a schematic diagram on a spectral curve before and after a refractive index adjustment of a first optical waveguide provided by the present application;

FIG. 8 illustrates a schematic structural diagram on a wearable medical device according to the present application;

Wherein: 1—light source; 2—single-ring shaped micro-ring resonant cavity; 21—first semi-circular cavity; 22—second semi-circular cavity; 3—annular mirror; 31—passive optical device; 32—loopback optical waveguide; 41—first phase-change device; 42—second phase-change device; 43—third phase-change device; 44—fourth phase-change device; 51—first coupler; 52—second coupler; 53—third coupler; 6—light absorber; 71—first optical waveguide; 72—second optical waveguide; 73—third optical waveguide; 74—fourth optical waveguide; 100—pump source; 200—prism coupling system; 300—crystal material; 400—lens system; 500—laser light source; 701—cladding layer; 702—waveguide core; 800—wearable medical device; 810—image acquisition unit; 811—photodetector; 812—amplifier; 813—filter; 814—signal processing circuit; 820—imaging unit; 821—display panel, 822—control circuit; 823—processor; 850—integrated laser.

### DESCRIPTION OF THE EMBODIMENTS

In order to make the purpose, technical solution and advantages of the present application clearer and more explicit, further detailed descriptions of the present application are stated here, referencing to the attached drawings and some embodiments of the present application. Obviously, the described embodiments are part of, but not all of, the embodiments of the present application. Based on the embodiments of the present application, all other embodiments obtained by those of ordinary skills in the art without any creative work are included in the scope of protection of the present application. Unless otherwise defined, technical or scientific terms used herein should have the meanings usually understood by those of ordinary skills in the art to which the present application belongs. As used herein, the terms “comprise” and the like are intended to mean that an element or item appearing before the term encompasses elements or items appearing after the term and the equivalents thereof, instead of excluding other elements or items.

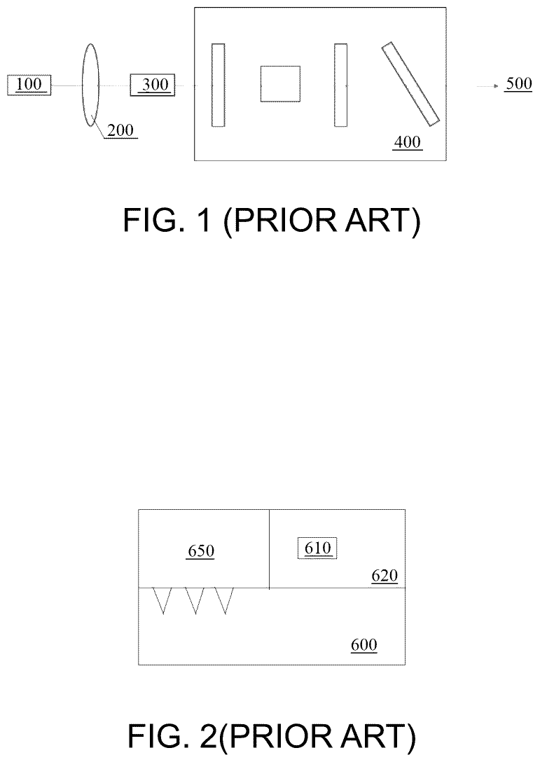

Shown as FIG. 1, in the prior art, a photon emitted by a pump source 100, passes through a prism coupling system 200 before entering a crystal material 300, after an interaction between the photon and the crystal material 300, the photon is then emitted out through a lens system 400, and acting as a laser light source 500.

Another case in the prior art is shown as FIG. 2, a material, such as Si 610 in groups III-V 650, such as SiO₂ 620, is integrated to a substrate of a silicon optical chip, Substrate 600, acting as a pump material, thereby achieving an integration between a laser and a silicon optical chip. However, such a technique cannot achieve an output wavelength tunable by the laser. In addition, due to silicon material has a high absorption loss in a wavelength range of 700-1200 nm, such a solution will limit a wavelength range of a laser light output from the silicon optical integrated laser.

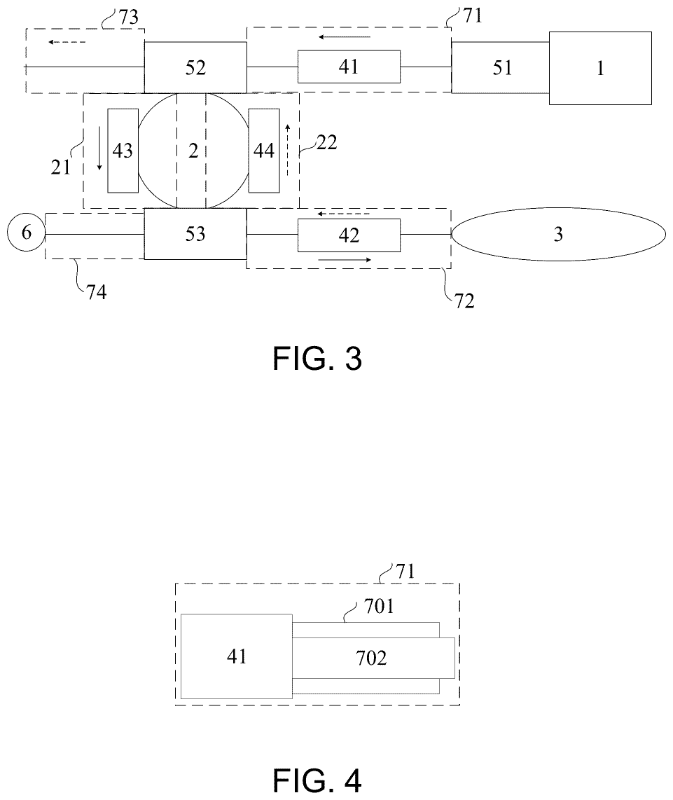

In view of the problems in the prior art, shown as FIG. 3, the present application provides an integrated laser, comprising: a light source 1, a resonant cavity, and an annular mirror 3; the light source 1 is connected to a head end of a first optical waveguide 71 through a first coupler 51; and a tail end of the first optical waveguide 71 is connected to a second coupler 52; the second coupler 52 is connected to a first end of the resonant cavity; the second coupler 52 further has a light output end arranged, configured to output a light wave input from the resonant cavity to an object; a second end of the resonant cavity is connected with a third coupler 53; the third coupler 53 is connected with the annular mirror 3 through a second optical waveguide 72; and at least one of the resonant cavity, the first optical waveguide 71 and the second optical waveguide 72 is made of a phase-change device.

Specifically, the resonant cavity is arranged as a non-linear closed-loop resonant cavity. a part of the first optical waveguide 71 is configured to be the first phase-change device 41, and a part of the second optical waveguide 72 is arranged as the second phase-change device 42. A wavelength range of the light emitted by the light source 1 covers an entire near-infrared wavelength range. The light source 1 may be configured as a common LED light source, and the light source 1, the resonant cavity and the annular mirror 3 are configured to construct a Fabry-Perot (F-P) laser locked in a high Q state. The high Q state refers to a state where a Q factor tends to infinity. The Q factor satisfies:

$$Q = \frac{{v}_{0}}{δ v}$$

Wherein v₀ represents a resonant frequency, and δv represents a half-height width of a resonant bandwidth. The light source 1 used in the present embodiment may adopt an F-P laser diode being locked in the high Q state by self-injection as the light source 1, and cooperate with a silicon optical chip, before being integrated into an external resonant cavity, it is possible to reduce a loss and an unnecessary reflection, thus a quality of an output light beam of the output optical signal is improved.

In a plurality of other embodiments, the second coupler 52 is further connected to a third optical waveguide 73, and the third optical waveguide 73 is configured to output an optical wave. In a plurality of more embodiments, the third coupler 53 is further connected a fourth optical waveguide 74, while another end of the fourth optical waveguide 74 is connected to a light absorber 6. In a plurality of other embodiments, there may not have the light absorber 6 arranged, and correspondingly, the fourth optical waveguide 74 is configured as a unidirectional optical waveguide, and the unidirectional optical waveguide only allows the optical wave to propagate in a direction away from the third coupler 53.

Shown as FIG. 4, in a plurality of other specific embodiments, the first optical waveguide 71 and the second optical waveguide 72 further comprises a waveguide core 702 and a cladding layer 701. The waveguide core 702 is arranged inside the cladding layer 701, and the waveguide core 702 is connected to the phase-change device. In an embodiment, the waveguide core 72 is made of a silicon nitride (SiN) material, the cladding layer 701 is made of a silicon oxide (SiO₂) material. The present embodiment arranges the waveguide core 702 by using a non-silicon crystal material, which is not sensitive to a temperature, thus able to avoid a defect that the output wavelength of a traditional laser is excessively affected by the temperature, and at a same time, an output wavelength range is not limited by an Si material.

In an embodiment, the resonant cavity is configured as a single-ring shaped micro-ring resonant cavity 2, comprising a first half-ring cavity 21 and a second half-ring cavity 22. The first half-ring cavity 21 is arranged as a third phase-change device 43, the second half-ring cavity 22 is arranged as a fourth phase-change device 44. It is worth noting that a diameter of the single-ring shaped micro-ring resonant cavity 2 is in an order of several microns to tens of microns, to ensure that the single-ring shaped micro-ring resonant cavity 2 can be manufactured by using a complementary metal-oxide-semiconductor (CMOS) process, which is convenient to be integrated into a silicon optical chip.

In another embodiment, the non-linear closed-loop resonant cavity is arranged in a closed loop having N circles rolling.

It should be noted that, by arranging at least one of the resonant cavity, the first optical waveguide 71 and the second optical waveguide 72 be made of a phase-change device, it is beneficial for realizing a refractive index change of an output light wave from the laser through an optical phase-change, and the wavelength of the output light wave is continuously tunable along with the refractive index, so as to obtain an output light wave with a required wavelength, which is beneficial to improving the precision of aiming a target tissue, avoiding an accidental injury to a surrounding normal tissue, and reducing a side effect.

In a plurality of embodiments, the third coupler 53 is further connected to a light absorber 6, configured to absorb a portion of the light wave output from the resonant cavity, so as to eliminate reflected light, and another part of the light wave output from the resonant cavity 2 is input into the annular mirror 3 by the second optical waveguide 7.

In an embodiment, a working bandwidth of the light absorber 6 is in an infrared band. In another embodiment, the working bandwidth of the light absorber 6 may be anyone wave band comprising an infrared band. It should be noted that a material of the light absorber 6 comprises germanium (Ge), indium phosphide (InP), and silicon (Si). In an embodiment, the light absorber 6 is made of silicon nitride. In another embodiment, the material of the light absorber 6 is configured as silicon oxynitride.

In a plurality of embodiments, the annular mirror 3 is configured to transmit the light wave input into the annular mirror back to the second optical waveguide 72; and the resonant cavity is configured to transmit the light wave input from the second optical waveguide 72 to the second coupler 52.

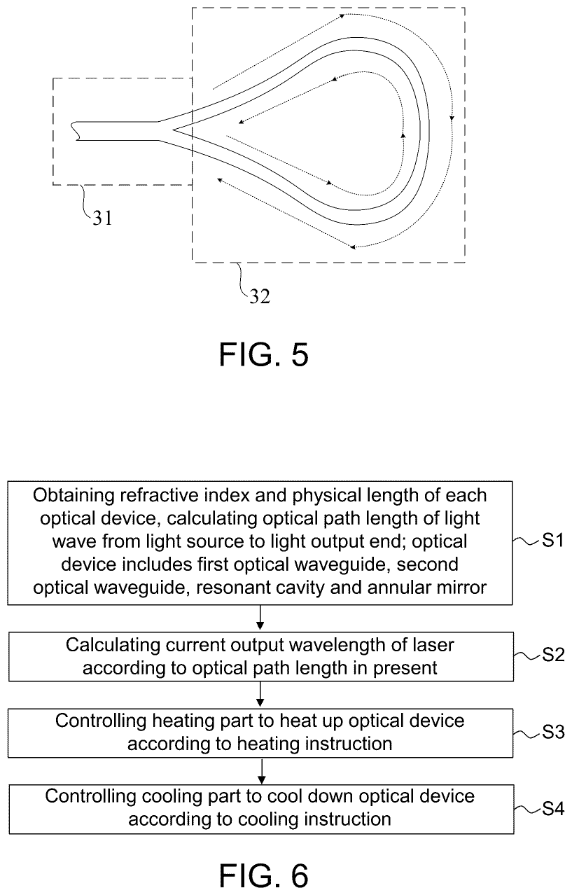

Shown as FIG. 5, specifically, the annular mirror 3 comprises a passive optical device 31 and a loopback optical waveguide 32; the passive optical device 31 has at least three terminals arranged, configured to split one light beam into two light beams; and the loopback optical waveguide 32 is connected to two of the at least three terminals of the passive optical device 31.

In an embodiment, the passive optical device 31 may be a Multi-Mode Interference Coupler (MMI), a Directional Coupler, (DC), a Y-splitter. The MMI may be configured as a type of single-input dual-output (1X2), or a type of dual-input dual-output (2X2). The DC may further comprise a Adiabatic Directional Coupler (ADC).

Referencing to FIG. 3 and FIG. 5, in an embodiment, the passive optical device 31 is a single-input dual-output multimode interference coupler, two output terminals thereof are respectively connected to a head end and a tail end of the loopback optical waveguide 32 to compose the annular mirror 3 in a water drop-shape, and the two output terminals have opposite optical paths arranged, shown as an anticlockwise path and a clockwise optical path in FIG. 5. The light wave input to the input terminal by the second optical waveguide 72 returns to the second optical waveguide 72 along the input terminal through the loopback optical waveguide 32.

In a plurality of embodiments, a lattice state of the phase-change device after being heated changes with a cooling speed, so that the refractive index of the phase-change device after being cooled down changes to a preset fixed value. It should be noted that, in the present embodiment, the lattice state of the phase-change device after being cooled down has already been fixed, and the refractive index thereof does not require a static power consumption to maintain, which is beneficial to saving power consumption. In the present embodiment, cooling down the phase-change device at a new speed after having been re-heated, it is possible to change the refractive index of the phase-change device conveniently, which is beneficial to improving a debugging efficiency.

Specifically, the phase-change device comprises an optical phase-change material (PCM). In an embodiment, the optical phase-change material comprises germanium antimony telluride (Ge₂Sb₂Te₅, TSG). In a plurality of other specific embodiments, the phase-change device may further comprise a solid-liquid phase-change material, a solid-gas phase-change material, and a solid-solid phase-change material.

Shown as FIG. 6, a second embodiment provides a wavelength control method for the integrated laser, configured to control the integrated laser according to any one of the embodiments stated above, comprising: S1, obtaining a refractive index and a physical length of each optical device, and calculating an optical path length of a light wave from the light source 1 to a light output end; the optical device comprises a first optical waveguide 71, a second optical waveguide 72, a resonant cavity and an annular mirror 3; S2, calculating a current output wavelength of the laser according to the optical path length in present; S3, controlling a heating part to heat up the optical device according to a heating instruction; and S4, controlling a cooling part to cool down the optical device according to a cooling instruction, while a cooling speed is adjustable.

In a plurality of embodiments, an output wavelength λ of the light wave satisfies:

$$m * λ = L$$

wherein L is the optical path length of the light wave from the light source 1 to the light output end, and m is a positive integer.

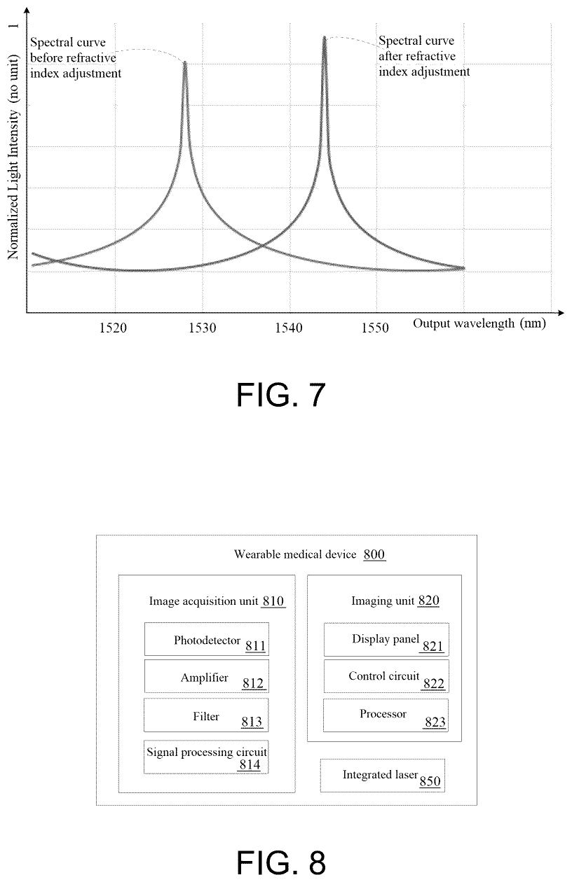

In a plurality of embodiments, the optical path length L satisfies:

$$L = {n}_{1} {l}_{1} + {n}_{2} {l}_{2} + … + {n}_{i} {l}_{i}$$

wherein i is a sequence number of each optical device in the laser; nᵢ is a refractive index of an i-th optical device; and lᵢ is a physical length of the i-th optical device. FIG. 7 shows a spectral curve before and after a refractive index adjustment of the first optical waveguide 71. It can be seen that an output power peak value of the laser is changed, and the output wavelength corresponding to the output power peak value is also changed.

In an embodiment, S3 further comprises adopting a tungsten (W) electrode to heat up the optical device. The tungsten electrode may be attached to the optical device, and configured to heat up the optical device when being connected to power. In another embodiment, an external laser may be adopted to irradiate the optical device, so as to heat up the optical device.

In a plurality of other embodiments, it is possible to adjust the refractive index of the optical device by controlling a pressure being applied to the optical device. In an embodiment, the optical device has a piezoelectric ceramic connected, and the piezoelectric ceramic is continuously powered. It is possible to achieve the pressure applied to the optical device by adjusting an input voltage of the piezoelectric ceramic.

Shown as FIG. 8, a third embodiment provides a wearable medical device 800, comprising the integrated laser 850 according to any one of the embodiments stated above.

Specifically, the wearable medical device 800 further comprises an image acquisition unit 810, configured to obtain a laser beam reflected by an object, and convert the laser beam into an electrical signal. In an embodiment, the image acquisition unit 810 comprises a photodetector 811, an amplifier 812, a filter 813, and a signal processing circuit 814. The object may be a tumor, a brain, a lymphatic vessel, and a gastrointestinal tract or any tissues.

In a plurality of other embodiments, the wearable medical device 800 further comprises an imaging unit 820, configured to convert the electrical signal having been acquired into a visible image. In an embodiment, the imaging unit 820 comprises a display panel 821, a control circuit 822, and a processor 823 configured to run an image processing algorithm.

While the embodiments of the present application have been described in detail above, it will be apparent to those skilled in the art that various modifications and variations can be made to the embodiments. It should be understood, however, that such modifications and variations are within the scope and spirit of the present application as set forth in the claims. Moreover, the present application described herein is capable of other embodiments and of being practiced or of being carried out in various ways.

What is claimed is:

1. An integrated laser, comprising a light source, a resonant cavity, and an annular mirror, wherein the resonant cavity comprises a single-ring shaped micro-ring resonant cavity, a first coupler, a second coupler, a third coupler, a first optical waveguide, and a second optical waveguide;
the single-ring shaped micro-ring resonant cavity comprises a first semi-circular cavity and a second semi-circular cavity; the first semi-circular cavity and the second semi-circular cavity are both configured as a phase-change device;
the light source is connected to a head end of the first optical waveguide through the first coupler;
a tail end of the first optical waveguide is connected to the second coupler;
the second coupler is connected to a first end of the single-ring shaped micro-ring resonant cavity;
the second coupler has a light output end, used to output a light wave input from the resonant cavity to an object;
a second end of the single-ring shaped micro-ring resonant cavity is connected with the third coupler; the third coupler is connected with the annular mirror through the second optical waveguide;
at least one of the first optical waveguide and the second optical waveguide has a portion configured as the phase-change device;
the phase-change device is made of an optical phase-change material;
when the first optical waveguide comprises the phase-change device, a waveguide core of the first optical waveguide is directly connected with the phase-change device, so as to allow light to be transmitted to directly pass through the optical phase-change material;
when the second optical waveguide comprises the phase-change device, a waveguide core of the second optical waveguide is directly connected with the phase-change device, so as to allow light to be transmitted to directly pass through the optical phase-change material;
the waveguide core is made of a silicon nitride material.

2. The integrated laser according to claim 1, wherein the third coupler is further connected to a light absorber, used to absorb a portion of the light wave output from the resonant cavity to eliminate reflected light;
another portion of the light wave output from the resonant cavity is input into the annular mirror by the second optical waveguide.

3. The integrated laser according to claim 2, wherein the annular mirror is configured to transmit the light wave input into the annular mirror back to the second optical waveguide;
the resonant cavity is configured to transmit the light wave input from the second optical waveguide to the second coupler.

4. The integrated laser according to claim 1, wherein the third coupler is further connected to a unidirectional optical waveguide, and the unidirectional optical waveguide allows a light wave to propagate in a direction away from the third coupler only.

5. The integrated laser according to claim 4, wherein the annular mirror is configured to transmit the light wave input into the annular mirror back to the second optical waveguide;
the resonant cavity is configured to transmit the light wave input from the second optical waveguide to the second coupler.

6. The integrated laser according to claim 1, wherein a lattice state of the phase-change device after being heated changes with a cooling speed, so that the refractive index of the phase-change device after being cooled down changes to a preset fixed value.

7. The integrated laser according to claim 1, wherein the resonant cavity is configured as a non-linear closed-loop resonant cavity.

8. The integrated laser according to claim 1, wherein the annular mirror comprises a passive optical device and a loopback optical waveguide;
the passive optical device has at least three terminals, configured to split one light beam into two light beams;
the loopback optical waveguide is connected to two of the at least three terminals of the passive optical device.

9. A wavelength control method configured to control the integrated laser according to claim 1, comprising:
S1, obtaining a refractive index and a physical length of each of a plurality of optical devices, and calculating an optical path length of a light wave from a light source to a light output end; each of the optical devices comprises a first optical waveguide, a second optical waveguide, a resonant cavity and an annular mirror;
S2, calculating a current output wavelength of the integrated laser according to the optical path length in present;
S3, controlling a heating part to heat up each of the optical devices according to a heating instruction; and
S4, controlling a cooling part to cool down each of the optical devices according to a cooling instruction, while a cooling speed is adjustable.

10. The wavelength control method according to claim 9, wherein the optical path length L satisfies:
$$L = {n}_{1} {l}_{1} + {n}_{2} {l}_{2} + … + {n}_{i} {l}_{i}$$
wherein i is a sequence number of each of the optical devices in the integrated laser; nᵢ is a refractive index of an i-th optical device; and lᵢ is a physical length of the i-th optical device.

11. The wavelength control method according to claim 9, wherein an output wavelength λ of the light wave satisfies:
$$m * λ = L$$
wherein L is the optical path length of the light wave from the light source to the light output end, and m is a positive integer.

12. The wavelength control method according to claim 11, wherein the optical path length L satisfies:
$$L = {n}_{1} {l}_{1} + {n}_{2} {l}_{2} + … + {n}_{i} {l}_{i}$$
wherein i is a sequence number of each of the optical devices in the integrated laser; nᵢ is a refractive index of an i-th optical device; and lᵢ is a physical length of the i-th optical device.

13. The wavelength control method according to claim 9, wherein the third coupler is further connected to a light absorber, configured to absorb a portion of the light wave output from the resonant cavity to eliminate reflected light;
another portion of the light wave output from the resonant cavity is input into the annular mirror by the second optical waveguide.

14. The wavelength control method according to claim 9, wherein the third coupler is further connected to a unidirectional optical waveguide, and the unidirectional optical waveguide allows a light wave to propagate in a direction away from the third coupler only.

15. A wearable medical device, comprising the integrated laser according to claim 1.
